(19) United States
(12) Patent Application Publication
(10) Pub. No.: US 2023/0030099 A1
(43) Pub. Date: Feb. 2, 2023

(54) PHARMACEUTICAL COMPOSITION COMPRISING MARMELO EXTRACT FOR PREVENTING OR TREATING OBESITY

(71) Applicant: BnG Inc., Chuncheon-si (KR)

(72) Inventor: Myeong Oh HWANG, Anyang-si (KR)

(21) Appl. No.: 17/535,815

(22) Filed: Nov. 26, 2021

(30) Foreign Application Priority Data
Jul. 9, 2021 (KR) 10-2021-0090503
Jul. 9, 2021 (KR) 10-2021-0090504
Jul. 9, 2021 (KR) 10-2021-0090505
Jul. 9, 2021 (KR) 10-2021-0090506

(51) Int. Cl. A61K 36/73; A61P 3/04

(52) U.S. Cl. CPC A61K 36/73; A61P 3/04

(57) ABSTRACT

The present invention relates to a composition for treatment and/or food usable in prevention, improvement and/or treatment of related disease such as reduction of body weight and/or body fat, lowering blood glucose, lowering cholesterol in blood, and the like, which comprises a marmelo extract as an active ingredient, and a method for preparation thereof.

### CROSS-REFERENCE TO RELATED APPLICATIONS

This application claims the benefits of Korean Application Nos. 10-2021-0090503, 10-2021-0090504, 10-2021-0090505, and 10-2021-0090506 filed on Jul. 9, 2021 with the Korean Intellectual Property Office, the entire disclosures of which are hereby incorporated by reference.

### TECHNICAL FIELD

The present invention relates to a composition for reducing body weight and/or body fat, a composition for lowering blood glucose, a composition for lowering cholesterol in blood and/or a composition for preventing, improving and/or treating of obesity and/or metabolic disease, which comprises a marmelo extract as an active ingredient.

### BACKGROUND ART

The obese population is rapidly increasing due to induction of excessive energy accumulation in the body by frequent consumption of instant food and meat-oriented diet and reduction of energy consumption by lack of exercise, and the like.

As such, excessively accumulated energy in the body appears in various disease forms as well as metabolic diseases including disease such as obesity, diabetes, hyperlipidemia and metabolic syndrome, and the like are representative.

Chlorogenic acid is one of the most consumed phenolic compounds in the human diet, and various studies have been conducted on the physiological activity of chlorogenic acid, and by these research results, various physiological activities of chlorogenic acid such as anti-oxidation, anti-inflammation, anti-diabetes and anti-obesity were confirmed.

The anti-obesity drugs and therapeutic agents for metabolic disease developed so far act on the sympathetic nerve or hormones and cause side effects, but there is a problem that the effect of herbal ingredients with few side effects is insignificant.

Accordingly, there is a need for development of a drug having excellent effects while having few side effects.

### PRIOR ART

### Patent Document

(Patent document 1) Korean patent publication No. 10-2015-0057119 (2015.05.28)

### DISCLOSURE

### Technical Problem

Under the above background, the present inventors repeated the study on the anti-obesity effect of chlorogenic acid. As a result, the content of chlorogenic acid in the marmelo extract was confirmed, and the weight gain inhibition, lipid metabolism improvement, obesity-related hormone level improvement, blood glucose reduction, blood cholesterol reduction, and the like of the marmelo extract comprising chlorogenic acid were confirmed, thereby completing the present invention.

One embodiment provides a composition for reducing body weight and/or body fat comprising a marmelo extract as an active ingredient.

Another embodiment provides a composition for lowering blood glucose comprising a marmelo extract as an active ingredient.

Other embodiment provides a composition for lowering cholesterol in blood comprising a marmelo extract as an active ingredient.

Other embodiment provides a composition for prevention, improvement and/or treatment of obesity comprising a marmelo extract as an active ingredient.

Other embodiment provides a composition for prevention, improvement and/or treatment of metabolic disease comprising a marmelo extract as an active ingredient.

The composition may be a pharmaceutical composition or food composition (for example, health functional food).

Other embodiment provides a method for reducing body weight comprising administering a pharmaceutically effective amount of marmelo extract to a subject in need of reduction of body weight and/or body fat.

Other embodiment provides a method for lowering body glucose comprising administering a pharmaceutically effective amount of marmelo extract to a subject in need of lowering blood glucose.

Other embodiment provides a method for lowering cholesterol in blood comprising administering a pharmaceutically effective amount of marmelo extract to a subject in need of lowering cholesterol in blood.

Other embodiment provides a method for prevention, improvement and/or treatment of obesity, comprising administering a marmelo extract to a subject in need of prevention, improvement and/or treatment of obesity.

Other embodiment provides a method for prevention, improvement and/or treatment of metabolic disease, comprising administering a marmelo extract to a subject in need of prevention, improvement and/or treatment of metabolic disease.

### Technical Solution

In the present description, it was confirmed that the marmelo extract administered with high-fat diet effectively inhibited the increase of body weight and/or body fat and fat tissue weight, blood glucose, blood cholesterol, and content of insulin and leptin in blood induced by the high-fat diet and inhibited the size increase of fat cells, thereby providing a use for anti-obesity, body weight and/or body fat reduction, blood glucose reduction, blood cholesterol reduction, prevention, improvement and/or treatment of metabolic disease of the marmelo extract.

Accordingly, one embodiment provides a composition for reducing body weight and/or body fat comprising a marmelo extract as an active ingredient.

Another embodiment provides a composition for lowering blood glucose comprising a marmelo extract as an active ingredient.

Other embodiment provides a composition for lowering cholesterol in blood comprising a marmelo extract as an active ingredient.

Other embodiment provides a composition for prevention, improvement and/or treatment of obesity comprising a marmelo extract as an active ingredient.

Other embodiment provides a composition for prevention, improvement and/or treatment of metabolic disease comprising a marmelo extract as an active ingredient.

The composition may be a pharmaceutical composition or food composition (for example, health functional food).

Other embodiment provides a method for reducing body weight and/or body fat comprising administering a pharmaceutically effective amount of marmelo extract to a subject in need of reduction of body weight.

Other embodiment provides a method for lowering body glucose comprising administering a pharmaceutically effective amount of marmelo extract to a subject in need of lowering blood glucose.

Other embodiment provides a method for lowering cholesterol in blood comprising administering a pharmaceutically effective amount of marmelo extract to a subject in need of lowering cholesterol in blood.

Other embodiment provides a method for prevention, improvement and/or treatment of obesity, comprising administering a marmelo extract to a subject in need of prevention, improvement and/or treatment of obesity. Other embodiment provides a method for prevention, improvement and/or treatment of metabolic disease, comprising administering a marmelo extract to a subject in need of prevention, improvement and/or treatment of metabolic disease.

Hereinafter, the present application will be described in more detail.

Marmelo Extract

In the present description, as an active ingredient for body weight and/or body fat reduction, blood glucose reduction, blood cholesterol reduction, prevention, improvement and/or treatment of obesity and/or metabolic disease, a marmelo extract is provided.

Marmelo (Cydonia oblonga) is a fruit tree of the Rosaceae family, also called quince, Aiva, or European quince, and has an appearance resembling the fruits of quince and apple, but is distinct from them.

The marmelo extract may be a solvent extract obtained by extracting the entire or partial (for example, fruit, seed, flesh, peel, leaf, stem, root, etc.) of marmelo using an extraction solvent as it is or in a dried and/or heated state.

The marmelo extract may be prepared by removing marmelo juice after compressing marmelo, and then and extracting the marmelo in the form of a solid remaining after with an extraction solvent, but not limited thereto.

In the present description, “marmelo juice” means a juice-type liquid generated by squeezing marmelo fruit.

The extraction solvent may be one or more kinds selected from the group consisting of water and a linear or branched chain alcohol having 1 to 4 carbon atoms (for example, ethanol, fermented spirits). In one specific embodiment, the extraction solvent may be one or more kinds selected from the group consisting of water and a linear or branched chain alcohol aqueous solution having 1 to 4 carbon atoms (for example, ethanol, fermented spirits), and the linear or branched chain alcohol aqueous solution having 1 to 4 carbon atoms may be for example, a linear or branched chain alcohol having 1 to 4 carbon atoms (for example, ethanol, spirits, fermented spirits) of 10 to 70 (v/v) %, 15 to 70 (v/v) %, 20 to 70 (v/v) %, 25 to 70 (v/v) %, 30 to 70 (v/v) %, 10 to 65 (v/v) %, 15 to 65 (v/v) %, 20 to 65 (v/v) %, 25 to 65 (v/v) %, 30 to 65 (v/v) %, 10 to 60 (v/v) %, 15 to 60 (v/v) %, 20 to 60 (v/v) %, 25 to 60 (v/v) %, 30 to 60 (v/v) %, 10 to 55 (v/v) %, 15 to 55 (v/v) %, 20 to 55 (v/v) %, 25 to 55 (v/v) %, 30 to 55 (v/v) %, 10 to 50 (v/v) %, 15 to 50 (v/v) %, 20 to 50 (v/v) %, 25 to 50 (v/v) % or 30 to 50 (v/v) %.

The amount of the extraction solvent used for extraction may be 1 to 10 volume times, 2 to 10 volume times, 3 to 10 volume times, 4 to 10 volume times, 1 to 9 volume times, 2 to 9 volume times, 3 to 9 volume times, 4 to 9 volume times, 1 to 8 volume times, 2 to 8 volume times, 3 to 8 volume times, 4 to 8 volume times, 1 to 7 volume times, 2 to 7 volume times, 3 to 7 volume times, 4 to 7 volume times, 1 to 6 volume times, 2 to 6 volume times, 3 to 6 volume times or 4 to 6 volume times of marmelo, but not limited thereto.

The extraction time is not limited thereto, but for sufficient extraction of the active ingredient, may be 1 to 24 hours, 1 to 12 hours, 1 to 6 hours, 1 to 5 hours, 3 to 24 hours, 3 to 12 hours, 3 to 6 hours, 3 to 5 hours, 5 to 24 hours, 5 to 12 hours or 5 to 6 hours. The extraction temperature is not limited thereto, but for effective extraction of the active ingredient, may be in a temperature range selected from the room temperature to the boiling point of the extraction solvent, for example, 50 to 100° C., 55 to 100° C., 60 to 100° C., 65 to 100° C., 50 to 95° C., 55 to 95° C., 60 to 95° C., 65 to 95° C., 50 to 90° C., 55 to 90° C., 60 to 90° C., 65 to 90° C., 50 to 85° C., 55 to 85° C., 60 to 85° C., 65 to 85° C., 50 to 80° C., 55 to 80° C., 60 to 80° C. or 65 to 80° C.

The extraction method for obtaining the extract may be selected from all known extraction methods commonly used in the art to which the present invention pertains. For example, commonly used extraction methods such as reflux extraction method, immersion extraction method, heat extraction method, enfleurage, hot water extraction method, ultrasonic extraction method, filtering method, pressurized extraction method, supercritical extraction method, electrical extraction method, and the like may be used.

The extraction may be performed once, or may be performed with different extraction solvents and/or conditions twice or more (for example, twice, 3 times, 4 times or 5 times). When the extraction is progressed twice or more, the extraction condition such as the extraction solvent used in each time, the extraction time per each time, temperature, and the like may be same or adjusted differently in the aforementioned range, but not limited thereto.

In one specific embodiment, the extract may have a sugar content of 10 to 100 brix, 20 to 100 brix, 30 to 100 brix, 40 to 100 brix, 50 to 100 brix, 60 to 100 brix, 70 to 100 brix, 80 to 100 brix, 90 to 100 brix, 10 to 90 brix, 20 to 90 brix, 30 to 90 brix, 40 to 90 brix, 50 to 90 brix, 60 to 90 brix, 70 to 90 brix, 80 to 90 brix, 10 to 80 brix, 20 to 80 brix, 30 to 80 brix, 40 to 80 brix, 50 to 80 brix, 60 to 80 brix, 70 to 80 brix, 10 to 70 brix, 20 to 70 brix, 30 to 70 brix, 40 to 70 brix, 50 to 70 brix or 60 to 70 brix, but not limited thereto.

In the present description, the marmelo extract may comprise not only the aforementioned solvent extract, but also a concentrate, a dried matter, and/or a crushed material, obtained by concentrating (for example, decompression concentration, vacuum concentration, etc.), drying (for example, spray drying, freeze-drying, etc.) and/or crushing (powdering) the solvent extract by a common method.

The marmelo extract provided as an active ingredient in the present description may have a chlorogenic acid content measured by HPLC of 0.2 mg/g or more, 0.3 mg/g or more, 0.4 mg/g or more, 0.5 mg/g or more, 0.6 mg/g or more or 0.7 mg/g or more, for example, 0.2 to 2 mg/g, 0.3 to 2 mg/g, 0.4 to 2 mg/g, 0.5 to 2 mg/g, 0.6 to 2 mg/g, 0.7 to 2 mg/g, 0.8 to 2 mg/g, 0.2 to 1.5 mg/g, 0.3 to 1.5 mg/g, 0.4 to 1.5 mg/g, 0.5 to 1.5 mg/g, 0.6 to 1.5 mg/g, 0.7 to 1.5 mg/g, 0.2 to 1.2 mg/g, 0.3 to 1.2 mg/g, 0.4 to 1.2 mg/g, 0.5 to 1.2 mg/g, 0.6 to 1.2 mg/g, 0.7 to 1.2 mg/g, 0.2 to 1.0 mg/g, 0.3 to 1.0 mg/g, 0.4 to 1.0 mg/g, 0.5 to 1.0 mg/g, 0.6 to 1.0 mg/g, 0.7 to 1.0 mg/g, 0.2 to 0.8 mg/g, 0.3 to 0.8 mg/g, 0.4 to 0.8 mg/g, 0.5 to 0.8 mg/g, 0.6 to 0.8 mg/g or 0.7 to 0.8 mg/g, based on the total weight of the extract (for example, concentrate weight).

Medical Use

One embodiment provides a pharmaceutical composition for body weight and/or body fat reduction comprising a marmelo extract as an active ingredient.

Another embodiment provides a pharmaceutical composition for lowering blood glucose comprising a marmelo extract as an active ingredient.

Other embodiment provides a pharmaceutical composition for lowering cholesterol in blood comprising a marmelo extract as an active ingredient.

Other embodiment provides a pharmaceutical composition for prevention, improvement and/or treatment of obesity comprising a marmelo extract as an active ingredient.

Other embodiment provides a pharmaceutical composition for prevention, improvement and/or treatment of metabolic disease comprising a marmelo extract as an active ingredient.

Other embodiment provides a method for reducing body weight comprising administering a pharmaceutically effective amount of a marmelo extract to a subject in need of body weight and/or body fat reduction.

Other embodiment provides a method for lowering blood glucose comprising administering a pharmaceutically effective amount of a marmelo extract to a subject in need of lowering blood glucose.

Other embodiment provides a method for lowering cholesterol in blood comprising administering a pharmaceutically effective amount of a marmelo extract to a subject in need of lowering cholesterol in blood.

Other embodiment provides a method for prevention, improvement and/or treatment of obesity comprising administering a pharmaceutically effective amount of a marmelo extract to a subject in need of prevention, improvement and/or treatment of obesity.

Other embodiment provides a method for prevention, improvement and/or treatment of metabolic disease comprising administering a pharmaceutically effective amount of a marmelo extract to a subject in need of prevention, improvement and/or treatment of metabolic disease.

The administering a pharmaceutically effective amount of the marmelo extract to a subject in need of (1) body weight and/or body fat reduction, (2) lowering blood glucose, (3) lowering cholesterol in blood and/or (4) prevention, improvement and/or treatment of obesity and/or metabolic disease may be administering the content of the marmelo extract of 1 to 400 mg/kg (body weight; BW), 50 to 400 mg/kg, 100 to 400 mg/kg, 150 to 400 mg/kg, 200 to 400 mg/kg, 1 to 300 mg/kg (body weight; BW), 50 to 300 mg/kg, 100 to 300 mg/kg, 150 to 300 mg/kg, 200 to 300 mg/kg, 1 to 200 mg/kg (body weight; BW), 50 to 200 mg/kg, 100 to 200 mg/kg, 150 to 200 mg/kg based on the body weight of the subject, but not limited thereto.

In the administering a pharmaceutically effective amount of the marmelo extract to a subject in need of (1) body weight and/or body fat reduction, (2) lowering blood glucose, (3) lowering cholesterol in blood and/or (4) prevention, improvement and/or treatment of obesity and/or metabolic disease, when the subject is a human, a dose may be determined by applying a human body conversion coefficient (0.08) and calculating. Accordingly, the administering to a human in need of prevention, improvement and/or treatment of obesity and/or metabolic disease may be administering the content of the marmelo extract of 0.0048 to 1.92 g/day, 0.24 to 1.92 g/day, 0.48 to 1.92 g/day, 0.72 to 1.92 g/day, 0.96 to 1.92 g/day, 0.0048 to 1.44 g/day, 0.24 to 1.44 g/day, 0.48 to 1.44 g/day, 0.72 to 1.44 g/day, 0.96 to 1.44 g/day, 0.0048 to 0.96 g/day, 0.24 to 0.96 g/day, 0.48 to 0.96 g/day or 0.72 to 0.96 g/day, based on the body weight 60 kg of the human to be administered, but not limited thereto.

The marmelo extract is as described above.

The lowering blood glucose refers to a concept including a decrease of the glucose concentration in blood, a decrease of an insulin content in blood, reduction of insulin resistance and/or an increase of insulin sensitivity. The insulin resistance may be calculated by the following Equation 2 of Example 6.3 and the insulin sensitivity may be calculated by the following Equation 3 of Example 6.3.

The lowering cholesterol in blood refers to reduction of the total cholesterol content or LDL (low-density lipoprotein) cholesterol content in blood.

The obesity generalizes a state in which body fat is abnormally large, and refers to a state in which a large amount of calories is ingested, but is not consumed, and is stored in the body in the form of fat.

In one embodiment, obesity may mean a state in which the body mass index (BMI; the value obtained by dividing the body weight by the square of the height; kg/m²) is about 25 kg/m² or more, but is not limited thereto.

The metabolic disease may be a generic term for disease that causes abnormalities in each organ as metabolites accumulate in the whole body or in a specific organ in the body due to a disorder or dysfunction of a body organ involved in hormones or metabolism.

In one embodiment, the metabolic disease may be disease induced by accumulation of one or more kinds selected from the group consisting of glucose, cholesterol, and triglyceride in blood or all of them. For example, the metabolic disease may be diabetes, hyperlipidemia, arteriosclerosis, hyperinsulinemia, or metabolic syndrome.

Diabetes is a type of systemic metabolic disease caused by genetic or environmental factors, and it is a disease caused by an absolute or relative lack of insulin in the body, and refers to a condition in which the blood glucose concentration is abnormally high. In the present description, diabetes may encompass all of type 1 diabetes, type 2 diabetes, and gestational diabetes, and in particular, it may be type 2 diabetes related to insulin resistance.

Hyperlipidemia may refer to a condition in which an excess of fat component is present in blood or causes inflammation. For example, when fasting serum cholesterol is 220 mg/dl or more or triglyceride is 150 mg/dl or more, hyperlipidemia may be diagnosed, but is not limited thereto. Hyperlipidemia may mean hypercholesterolemia, hypertriglyceridemia or both.

Arteriosclerosis is a generic term for disease in which blood vessels in the arteries are narrowed due to formation of blood clots in the arteries, and in the present description, it may be related to high cholesterol and/or triglyceride content in blood.

Hyperinsulinemia refers to a condition in which the blood insulin level is high, and it may be divided into organic hyperinsulinemia caused by proliferation (adenoma, hypertrophy) of Langerhans islets, or functional hyperinsulinemia caused by dysfunction of autonomic nervous system and digestive system without adenoma of Langerhans islets.

Metabolic syndrome refers to a syndrome in which risk factors such as hyperlipidemia, high blood pressure, abnormal glucose metabolism, and obesity appear together. Metabolic syndrome is a disease presumed to be caused by insulin resistance and may mean symptoms in which two or more of cholesterol, blood pressure and blood glucose level are abnormal.

In the present description, ‘treatment’ is used in meaning which includes all of improvement, alleviation or stabilization of disease state or symptoms, partial or complete recovery, prolongation of survival, reduction of disease range, delay or alleviation of disease progression, and other beneficial treatment results, and the like. ‘Prevention’ is used in meaning which includes all mechanisms and/or effects of preventing the onset of a specific disease, delaying the onset of the disease, or lowering the frequency of onset, by acting on a subject not having a specific disease.

The content of the marmelo extract used as an active ingredient in the pharmaceutical composition provided in the present description may be suitably adjusted depending on the type and purpose of use, the condition of the subject, the type and severity of symptoms, and the like, and it may be 0.001 to 99.9% by weight, 0.001 to 90% by weight, 0.001 to 75% by weight, 0.001 to 50% by weight, 0.01 to 99.9% by weight, 0.01 to 90% by weight, 0.01 to 75% by weight, 0.01 to 50% by weight, 0.1 to 99.9% by weight, 0.1 to 90% by weight, 0.1 to 75% by weight, 0.1 to 50% by weight, 1 to 99.9% by weight, 1 to 90% by weight, 1 to 75% by weight, 1 to 50% by weight, 5 to 99.9% by weight, 5 to 90% by weight, 5 to 75% by weight, 5 to 50% by weight, 10 to 99.9% by weight, 10 to 90% by weight, 10 to 75% by weight, or 10 to 50% by weight, based on the solid (solid component from which the solvent is removed from the extract) weight, but not limited thereto.

The pharmaceutical composition or marmelo extract as an active ingredient may be administered to a subject for administration selected from vertebrates including primates such as humans, monkeys, rodents such as mice, rats and rabbits, other mammals such as dogs, cats, cattle, pigs, sheep, horses, goats, birds such as chickens, ducks, geese, reptiles such as snakes, lizards, turtles, crocodiles, amphibians and fish, and the like through various routes. The administration method of the pharmaceutical composition may be all the commonly used methods, and for example, it may be administered by a route of oral administration or parenteral administration such as intravenous, intramuscular, subcutaneous or intraperitoneal injection. In one embodiment, the pharmaceutical composition may be for oral administration, but not limited thereto.

The pharmaceutical composition may be used by formulating into an oral formulation such as powder, granules, tablets, capsules, suspension, emulsion, syrup, aerosol, or a parenteral formulation in a sterile injection solution form according to common methods, respectively.

The pharmaceutical composition may be administered as mixed with one or more adjuvants selected from the group consisting of pharmaceutically and/or physiologically acceptable carriers, excipients and diluents, selected generally in consideration of administration methods and Standard pharmaceutical practice. For example, the pharmaceutically and/or physiologically acceptable carrier may comprise one or more kinds selected from the group consisting of lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil, and the like, but not limited thereto, and may be all carriers commonly used in the pharmaceutical field.

The pharmaceutically and/or physiologically acceptable diluent and/or excipient may be one or more kinds selected from the group consisting of all fillers, extenders, binders, wetting agents, disintegrating agents, lubricants, surfactants, and like, commonly used in appropriate formulation of the pharmaceutical composition.

For example, in case of solid formulations for oral administration such as tablets, pills, powder, granules or capsules, the excipient may be at least one or more kinds of substances for formulation of this solid formulation, for example, one or more kinds selected from the group consisting of starch, calcium carbonate, sucrose, lactose, gelatin, and the like. In addition, in case of solid formulations for oral administration such as suspensions, internal solutions, emulsions, or syrups, it may be at least one or more selected from the group consisting of water, liquid paraffin, and the like, which are commonly used simple diluents, in addition thereto, lubricants such as magnesium stearate, talc and the like may be used. In addition, various excipients commonly used, for example, wetting agents, sweeteners, flavoring agents and/or preservatives, and the like may be comprised, but not limited thereto.

For example, the pharmaceutical composition may be administered orally, intraorally, or sublingually, in a table form containing starch or lactose, or a capsule form containing an appropriate excipient, or an elixir or suspension form containing a flavoring or coloring chemical. This liquid formulation may be formulated with a pharmaceutically acceptable additive such as suspension (for example, semisynthetic glyceride such as methyl cellulose, Witepsol, or glyceride mixture such as a mixture of apricot kernel oil and PEG-6 ester or a mixture of PEG-8 and caprylic/capric glyceride), but not limited thereto.

Food Composition

One embodiment provides a food composition for body weight and/or body fat reduction comprising a marmelo extract as an active ingredient.

Another embodiment provides a food composition for lowering blood glucose comprising a marmelo extract as an active ingredient.

Other embodiment provides a food composition for lowering cholesterol in blood comprising a marmelo extract as an active ingredient.

Other embodiment provides a food composition for prevention and/or improvement of obesity comprising a marmelo extract as an active ingredient.

Other embodiment provides a food composition for prevention and/or improvement of metabolic disease comprising a marmelo extract as an active ingredient.

Other embodiment provides a method for lowering body weight comprising administering a food composition comprising a marmelo extract as an active ingredient to a subject in need of (1) body weight and/or body fat reduction, (2) lowering blood glucose, (3) lowering cholesterol in blood and/or (4) prevention, improvement and/or treatment of obesity and/or metabolic disease.

The administering the food composition into a subject in need of (1) body weight and/or body fat reduction, (2) lowering blood glucose, (3) lowering cholesterol in blood and/or (4) prevention, improvement and/or treatment of obesity and/or metabolic disease may administer the content of the marmelo extract of 1 to 400 mg/kg (body weight; BW), 50 to 400 mg/kg, 100 to 400 mg/kg, 150 to 400 mg/kg, 200 to 400 mg/kg, 1 to 300 mg/kg (body weight; BW), 50 to 300 mg/kg, 100 to 300 mg/kg, 150 to 300 mg/kg, 200 to 300 mg/kg, 1 to 200 mg/kg (body weight; BW), 50 to 200 mg/kg, 100 to 200 mg/kg, 150 to 200 mg/kg, based on the body weight of the subject, but not limited thereto.

In the administering the food composition into a subject in need of (1) body weight and/or body fat reduction, (2) lowering blood glucose, (3) lowering cholesterol in blood and/or (4) prevention, improvement and/or treatment of obesity and/or metabolic disease, when the subject is a human, the dose may be determined by calculating by applying a human conversion factor (0.08). Accordingly, the administering to a human in need of prevention, improvement and/or treatment of obesity and/or metabolic disease may administer the content of the marmelo extract of 0.0048 to 1.92 g/day, 0.24 to 1.92 g/day, 0.48 to 1.92 g/day, 0.72 to 1.92 g/day, 0.96 to 1.92 g/day, 0.0048 to 1.44 g/day, 0.24 to 1.44 g/day, 0.48 to 1.44 g/day, 0.72 to 1.44 g/day, 0.96 to 1.44 g/day, 0.0048 to 0.96 g/day, 0.24 to 0.96 g/day, 0.48 to 0.96 g/day or 0.72 to 0.96 g/day, based on the body weight 60 kg of the human, but not limited thereto.

The marmelo extract, lowering blood glucose, lowering cholesterol in blood and metabolic disease are as described above.

In the food composition provided in the present description, the term “food” means an edible natural product or processed product containing one or more nutrients, and as a common meaning, it may be used to mean one or more selected from the group consisting of various kinds of general food, health functional food, beverages, food additives, beverage additives, and the like. The term “food composition” may mean a combination of raw materials for preparing the food. In one embodiment, the food may mean health functional food.

The content of the marmelo extract in the food composition may be appropriately adjusted according to the food use form, purpose, and the like, and for example, it may be 0.00001 to 99.9% by weight, 0.0001 to 99.9% by weight, 0.001 to 99.9% by weight, or 0.001 to 50% by weight, based on the solid weight, but not limited thereto.

The administration ‘subject’ of the food composition may be a subject selected from vertebrates including primates such as humans, monkeys, rodents such as mice, rats and rabbits, other mammals such as dogs, cats, cattle, pigs, sheep, horses, goats, birds such as chickens, ducks, geese, reptiles such as snakes, lizards, turtles, crocodiles, amphibians and fish, and the like, a cell or tissue isolated from the subject, or cell culture, or the like.

### Advantageous Effects

The marmelo extract provided herein has an anti-obesity effect against dietary obesity and a lipid improvement effect by reducing body weight and/or body fat and fat tissue weight, blood glucose, cholesterol in blood, and insulin and leptin in blood or inhibiting their increase effectively, and furthermore, it suggests that there is an effect of prevention, treatment and/or improvement of metabolic disease, and it suggests the possibility of development as a health functional material having an anti-obesity efficacy and/or an effect of prevention, treatment and/or improvement of metabolic disease hereafter.

### BRIEF DESCRIPTION OF THE DRAWINGS

FIG. 1a and FIG. 1B are graphs showing the result of analyzing the chlorogenic acid content in the marmelo extract by HPLC chromatography. FIG. 1a shows the chlorogenic acid standard product, and FIG. 1B shows the result of Sample c (Table 4 of Example 2.2).

FIG. 2 is a graph showing the weekly body weight change according to the diet type. Group 1 refers to the control diet group, and Group 2 refers to the high-fat diet group, and Group 3 refers to the high-fat diet group administered with mdB-44.

FIG. 3 is a photograph observing the histological change of the epididymis fat tissue using an optical microscope (Carl Zeiss).

FIG. 4 is a graph showing the fat accumulation inhibitory effect by confirming the fat accumulation amount according to the extraction method of the marmelo extract. The first—is without the addition of an extract and no differentiation, the second—is for differentiation and culture without adding an extract, EE is a marmelo extract extracted with ethanol without additional processing, and mdB-44 is a marmelo extract with additional processing.

### MODE FOR INVENTION

Hereinafter, the present invention will be described in more detail by examples. However, the following examples are intended to illustrate the present invention only, but the scope of the present invention is not limited by the following examples.

### Example 1. Preparation of Marmelo Extract

Marmelo fruits (LICORICE EXTRACT (Tashkent, Republic of Uzbekistan)) were weighed, selected, washed and foreign substances were removed. Then, the marmelo was compressed with a compressor at a pressure of 1˜5 kg/cm² to remove marmelo juice (liquid in a juice form generated by compressing marmelo fruits) and isolate solids. The solids were extracted with 30˜50 (v/v) % spirit of 4˜6 volume times at 65˜80° C. for 5˜6 hours twice. The extract was filtered with a 10 μm standard filter, and vacuum concentrated to 65 brix or more using a vacuum evaporator (EYELA, Tokyo, Japan) to obtain a marmelo concentrate. The concentrate was powdered by a spray drier. The marmelo extract in a concentrate or powder phase prepared as above was used for the following example.

### Example 2. Analysis of Chlorogenic Acid Content of Marmelo Extract

2.1 HPLC Analysis Method

The chlorogenic acid (CGA) content of the marmelo extract prepared in Example 1 was analyzed using LC-20A high-performance liquid chromatography (HPLC) system (Shimadzu, Japan). The HPLC analysis conditions were shown in the following Table 1 (analysis conditions), and Table 2 (moving bed gradient condition).

TABLE 1

| Items | Conditions |
|---|---|
| Column | Capcell Pak C₁₈ column |
| | (Shiseido, 5 μm, 250 × 4.6 mm) |
| Column Temperature | 40° C. |
| Flow | 1.4 mL/min |
| Detector | Diode Array Detector (DAD) 330 nm |
| Injection Volume | 5 μL |
| Mobile Phase | A: phosphoric acid:water = 0.5:99.5(v/v), |
| | B: phosphoric acid:AcN = 0.5:99.5(v/v) |
| Run time | 40 min |

TABLE 2

| Time (Min) | Solvent A (%) | Solvent B (%) |
|---|---|---|
| 0 | 95 | 5 |
| 7 | 95 | 5 |
| 27 | 70 | 30 |
| 28 | 10 | 90 |
| 30 | 10 | 90 |
| 31 | 95 | 5 |
| 40 | 95 | 5 |

Chlorogenic acid standard product 5 mg was weighed and dissolved in methanol 50 mL. 5 mL was taken in this solution and moved to a volume flask, and 5 mL methanol was added and 20 mL distilled water was added to use it as a standard solution stock solution. The solution of the stock solution was used as a standard stock solution, and was diluted step by step to prepare a calibration curve to make a standard solution for chlorogenic acid (final concentration: 0.00833, 0.00416, 0.00208, 0.00104, 0.01666 mg/mL), and the corresponding information was shown in the following Table 3. In addition, the marmelo extract 30 mg in a powder state of Example 1 was weighed and dissolved by adding 30% methanol 5 mL and filtered with a 0.45 um filter and used as a test solution (final concentration 30 mg/5 mL) (marmelo extract). The corresponding information was shown in the following Table 3.

TABLE 3

| Sample | Pretreatment concentration (Concentration) |
|---|---|
| Chlorogenic acid standard | 0.00833, 0.00416, 0.00208, |
| solution | 0.00104, 0.01666 mg/mL in |
| | 30% MeOH |
| Marmelo extract | 30 mg/5 mL in 30% MeOH (HPLC grade) |

2.2 Experimental Result

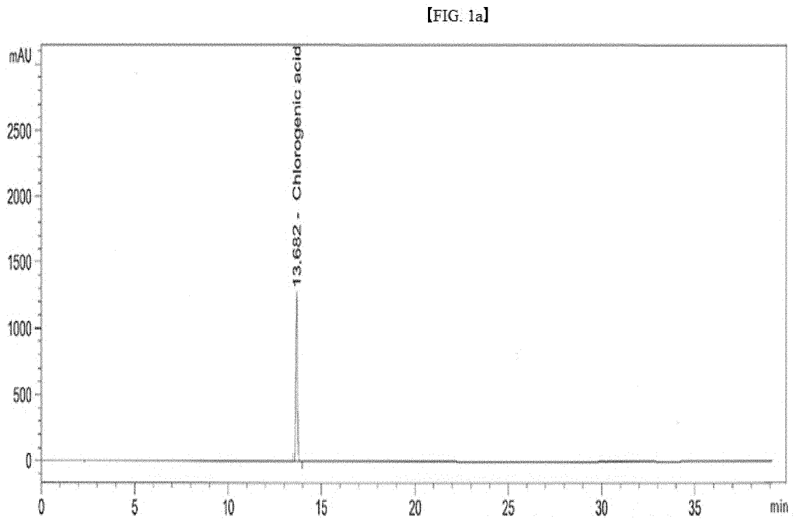

By the HPLC analysis method of 2.1, an experiment was performed three times, and the obtained values were shown in Table 4, FIGS. 1a (chlorogenic acid Standard product) and 1b (Sample c).

TABLE 4

| Sample name | Content (mg/g) |
|---|---|
| Sample a | 0.82 |
| Sample b | 0.7 |
| Sample c | 0.73 |

(The Samples a, b and c mean test solutions including the marmelo extract used for each experiment, when performing the experiment 3 times)

As shown in Table 4, FIG. 1a and FIG. 1B, the chlorogenic acid content in the marmelo extract prepared in Example 1 was shown as 0.82 mg/g in Sample a, 0.7 mg/g in Sample b, and 0.73 mg/g in Sample c, and therefore, it was confirmed that the distribution of the chlorogenic acid content was in the range of ±20% based on the case of Sample c, which had an intermediate value. The result shows that the marmelo extract obtained in Example 1 comprises chlorogenic acid in a certain range (for example, 0.75(mg/g)±20%) and this result shows that the chlorogenic acid content comprised in the extract is in an equal range even after performing the extraction several times.

### Example 3. Preparation of Test Material and Experimental Animal

3.1 Test Material Preparation and Animal Experiment Approval

As a test material, the marmelo extract in a powder form prepared in Example 1 (powder: hereinafter, ‘mdB-44’) was used, and all the animal experiments in the following examples were performed in accordance with the animal experiment regulations under the approval of the Animal Experiment Ethics Committee of Hallym University (Hallym 2019-67).

3.2 Test Group and Test Material Administration

Specific pathogen free 5-week-old, male C57BL/6 mice were purchased from Doo Yeol Biotech and used. After one week of quarantine and adaptation, healthy animals without weight loss were selected and used for the experiment. The experiment animals were bred in a breeding environment set at a temperature of 23±3° C., a relative humidity of 50±10%, a ventilation frequency of 10˜15 times/hour, an illumination time of 12 hours (08:00˜20:00), and an illumination intensity of 150˜300 Lux. Adaptation period experimental animals were allowed to freely ingest solid feed for laboratory animals (Cargill Agripurina, Co., Ltd.) and drinking water. After a one-week adaptation period, healthy animals were selected and were classified into the control diet group (CD) (Group 1), high-fat diet (HFD) control group (Group 2), high-fat diet+200 mg/kg body weight (BW) mdB-44 (experimental animals of Example 3.1) administration group (Group 3) by randomized block design. The energy ratio (kcal %) (protein:carbohydrate:fat) of the control diet for the whole test period was 20:70:10, and the energy ratio (kcal %) (protein:carbohydrate:fat) of the high-fat diet was 20:20:60, and both diets were purchased from Research Diets, Inc. (New Brunswick, N.J., USA). The composition of the control diet group and high-fat diet was shown in Table 5, and the experiment design was shown in Table 6.

TABLE 5

| | | Control diet | | High-fat diet |
|---|---|---|---|---|
| | Control diet | (CD) (10 | High-fat diet | (HFD) (60 |
| | (CD) (10 | kcal % fat) | (HFD) (60 | kcal % fat) |
| | kcal % fat) | each | kcal % fat) | each |
| | each | composition | each | composition |
| | composition | calorie content | composition | calorie content |
| | content (g) | (kcal) | content (g) | (kcal) |
| Casein, 80 Mesh | 200 | 800 | 200 | 800 |
| L-Cystine | 3 | 12 | 3 | 12 |
| Corn starch | 315 | 1260 | 0 | 0 |
| Maltodextrin 10 | 35 | 140 | 125 | 500 |
| Sucrose | 350 | 1400 | 68.8 | 275.2 |
| Cellulose, BW200 | 50 | 0 | 50 | 0 |
| Soybean oil | 25 | 225 | 25 | 225 |
| Lard* | 20 | 180 | 245 | 2205 |
| Mineral mix | 10 | 0 | 10 | 0 |
| Dicalcium phosphate | 13 | 0 | 13 | 0 |
| Calcium carbonate | 5.5 | 0 | 5.5 | 0 |
| Potassium citrate, | 16.5 | 0 | 16.5 | 0 |
| 1H₂O | | | | |
| Vitamin mix | 10 | 40 | 10 | 40 |
| V10001 | | | | |
| Choline bitartrate | 2 | 0 | 2 | 0 |
| Total | 1055.05 | 4057 | 773.85 | 4057 |

*(Lard: calculating cholesterol as 0.95 mg/g)

TABLE 6

| | Group 1 | Group 2 | Group 3 |
|---|---|---|---|
| | Control group | Negative control | Experimental |
| | (normal) | group | group |
| Experimental diet | CD | HFD | HFD |
| Test material | — | — | mdB-44 200 |
| (mg/kg BW) | | | |
| Number of | 10 | 10 | 10 |
| animals (head) | | | |

During the entire period of the test, drinking water was freely ingested, and 200 mg of the test material of Example 3.1 was dissolved in the drinking water per 1 kg and orally administered at a certain time for 8 weeks (Group 3). In the following example using the corresponding experimental animal, statistical processing was expressed as mean±standard error. The collected result was analyzed using GraphPad Prism 5.0 (GraphPad software) program. To compare the difference between the test material administration group and control group, Student's t-test and one-way analysis variance (ANOVA) were used. It was judged to be statistically significant only when p<0.05 or more.

### Example 4. Body Weight and Dietary Intake Measurement

The body weight of the experiment animals prepared in Example 3 at week 0 and week 8, and dietary intake was measured at 2-day intervals for 8 weeks. The amount ingested during the entire period of the test was calculated to calculate the 1-day dietary intake and 1 day energy intake. Food Efficiency Ratio (FER) was calculated as in Equation 1 below by dividing the weight gain during the test period by the amount of food consumed during the same period.

Food Efficiency Ratio=body weight gain (g)/dietary intake (g) [Equation 1]

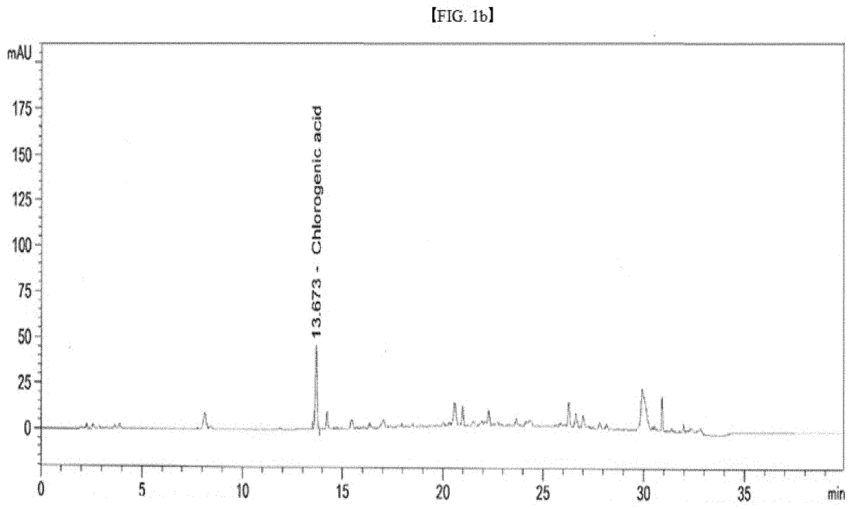

The measured body weight result and the total body weight gain and daily body weight gain according thereto were shown in Table 7 and the dietary intake and food efficiency ratio measured for 8 weeks were shown in Table 8, and in particular, the body weight result was shown in FIG. 2.

TABLE 7

| | Group 1 | Group 2 | Group 3 |
|---|---|---|---|
| Experiment | CD | HFD | HFD |
| diet | | | |
| Test material | — | — | mdB-44 200 |
| (mg/kg BW) | | | |
| Week 0 | 23.14 ± 0.35 | 23.06 ± 0.24 | 23.10 ± 0.27 |
| Week 8 | 31.20 ± 0.67 | 41.91 ± 0.91*** | 36.45 ± 0.91^{###} |
| Total body | 8.1 ± 0.7 | 18.9 ± 0.9*** | 13.4 ± 1.1^{##} |
| weight gain (g) | | | |
| Daily body | 0.146 ± 0.013 | 0.343 ± 0.016*** | 0.243 ± 0.020^{##} |
| weight gain | | | |
| (g/day) | | | |

(The result in Table 8 is expressed as mean ± standard error, and means
*p < 0.05,
**p < 0.01,
***p < 0.001,
^{#}p < 0.05,
^{##}p < 0.01,
^{###}p < 0.001;
Group 1 (normal control group);
Group 2 (negative control group);
Group 3: experimental group, (mdB-44 administration group)

TABLE 8

| | Group 1 | Group 2 | Group 3 |
|---|---|---|---|
| Experiment diet | CD | HFD | HFD |
| Test material (mg/kg BW) | — | — | mdB-44 200 |
| Total diet intake (g) | 145.4 ± 1.4 | 129.1 ± 1.2*** | 112.7 ± 1.5^{###} |
| Daily diet intake (g/day) | 2.64 ± 0.03 | 2.35 ± 0.02*** | 2.05 ± 0.03^{###} |
| Daily energy intake (kcal/day) | 10.18 ± 0.10 | 12.30 ± 0.11*** | 10.73 ± 0.15^{###} |
| Food efficiency ratio (body | 0.055 ± 0.005 | 0.146 ± 0.007*** | 0.119 ± 0.009^{#} |
| weight gain/diet intake) | | | |

(The numerical value is expressed as mean ± standard error, and means
*p < 0.05,
**p < 0.01,
***p < 0.001,
^{#}p < 0.05,
^{##}p < 0.01,
^{###}p < 0.001)

As shown in Tables 7 and 8 above, the experimental animals of all the test groups showed a normal change in body weight as their body weight continued to increase during the test period. Compared to the control diet group (Group 1), the body weight of the high-fat diet control group (Group 2) was significantly increased. On the other hand, in Group 3, the test group to which the marmelo extract was administered, compared to the high-fat diet control group (Group 2), the body weight of the experimental animals increased by high-fat diet supply (body weight gain) was significantly reduced. Through this, it was confirmed that even with a high-fat diet, when the marmelo extract was consumed in parallel, weight gain was suppressed.

In addition, the dietary intake of Group 3, the test group to which the marmelo extract was administered was significantly reduced, compared to the negative control group (Group 2), but the food efficiency ratio (body weight gain/dietary intake) was also reduced, and this means that weight loss is not due to a decrease in dietary intake alone.

### Example 5. Body Fat Measurement

After anesthetizing the experimental animals 1 day before completing the experimental animal test of Example 3.2, body fat percentage was evaluated by measuring body components using dual-energy X-ray absorptiometry (DEXA, PIXImus™, GE Lunar), and the result was shown in Table 9.

TABLE 9

| | Group 1 | Group 2 | Group 3 |
|---|---|---|---|
| Experiment diet | CD | HFD | HFD |
| Test material | — | — | mdB-44 200 |
| (mg/kg BW) | | | |
| Fat (%) | 29.58 ± 0.84 | 42.84 ± 1.66*** | 37.97 ± 1.46^{#} |

(The numerical value is expressed as mean ± standard error, and means
*p < 0.05,
**p < 0.01,
***p < 0.001,
^{#}p < 0.05,
^{##}p < 0.01,
^{###}p < 0.001)

In the body fat percentage, compared to 29.58±0.84% of the control diet group (Group 1), the body fat percentage was significantly increased as 42.84±1.66% in the high-fat diet control group (Group 2), and compared to the high-fat diet control group (Group 2), the body fat percentage was significantly reduced in the mdB-44 200 mg/kg BW administration group (Group 3).

### Example 6. Blood and Fat Analysis

### Example 6.1 Blood Collecting and Tissue Extraction

After completion of the test of the experimental animal of Example 3, the experimental animal was sacrificed. Before sacrifice, after anesthetizing using an anesthetic prepared by diluting tribromoethanol with tert-amyl alcohol, orbital blood was collected. Blood was placed in a serum separate tube (Becton Dickinson), left at a room temperature for 30 minutes, and then centrifuged at 5,000 rpm for 10 minutes to separate serum, and stored at −70° C. until analysis. After blood collection, the experimental animal was sacrificed and white fat tissue (epididymal fat, visceral fat, retroperitoneal fat, inguinal fat) was extracted, and then rinsed with cold physiological saline, and excess water was removed with filter paper, and the weight was measured. Epididymal fat tissue was divided into thirds and part was fixed in 4% paraformaldehyde (PFA) and then embedded in paraffin to perform tissue staining, for a part, total RNA was isolated and then real-time PCR was performed, and for a part, protein was isolated and then western blot was performed, and it was stored at ˜70° C. for following further analysis.

6.2 Tissue (Fat Tissue) Weight Measurement

The weight of the fat tissue in Example 6.1 was measured, and the result was shown in Table 10.

TABLE 10

| | Group 1 | Group 2 | Group 3 |
|---|---|---|---|
| Experiment diet | CD | HFD | HFD |
| Test material (mg/kg BW) | — | — | mdB-44 200 |
| Epididymal fat | 1.030 ± 0.070 | 2.471 ± 0.079*** | 2.023 ± 0.109^{##} |
| Retroperitoneal fat | 0.447 ± 0.037 | 1.184 ± 0.063*** | 0.954 ± 0.074^{#} |
| Visceral fat | 0.603 ± 0.050 | 1.603 ± 0.114*** | 0.975 ± 0.073^{###} |
| Inguinal fat | 0.210 ± 0.031 | 0.544 ± 0.033*** | 0.490 ± 0.052 |
| White fat total weight | 2.29 ± 0.14 | 5.80 ± 0.21*** | 4.44 ± 0.21^{###} |

(The numerical value is expressed as mean ± standard error, and means
*p < 0.05,
**p < 0.01,
***p < 0.001,
^{#}p < 0.05,
^{##}p < 0.01,
^{###}p < 0.001)

Body fat is classified into white fat tissue and brown fat tissue according to its shape and action. White fat tissue mainly stores excess energy in the body as fat, and brown fat tissue functions to produce heat and as the weight of white fat increases, the body weight increases. The weight of all of the epididymal fat, retroperitoneal fat, visceral fat and inguinal fat which are white fat tissue, was significantly increased in the high-fat diet control group (Group 2) compared to the control diet group (Group 1). On the other hand, in Group 3 to which the marmelo extract was administered, compared to the high-fat diet control group (Group 2), the body fat of all the tested items was reduced.

6.3 Blood Glucose-Related Numerical Value Analysis

The content of glucose in blood collected in Example 6.1 was measured using a blood biochemical analyzer (KoneLab 20 XT, Thermo Fisher Scientific), and the insulin content was measured using a measurement kit purchased from Millipore Corporation according to the method suggested by the manufacturer.

Since insulin resistance is the most important factor in metabolic syndrome, measurement of blood insulin concentration is very useful for measuring insulin sensitivity and resistance, and in the present study, insulin resistance (HOMA-IR) and insulin sensitivity (QUICKI) were calculated using the blood insulin concentration and fasting blood glucose. Insulin resistance (homeostatic model assessment for insulin resistance, HOMA-IR) and insulin sensitivity (quantitative insulin sensitivity check index, QUICKI) were calculated using the fasting blood glucose and blood insulin content by the following Equations 2 and 3, and the measured result was shown in Table 11.

Insulin resistance (HOMA-IR)=[insulin (mU/L)×glucose (mg/dL)]/405 [Equation 2]

Insulin sensitivity (QUICKI)=1/[log insulin (mU/L)+log glucose (mg/dL)] [Equation 3]

TABLE 11

| | Group 1 | Group 2 | Group 3 |
|---|---|---|---|
| Experiment diet | CD | HFD | HFD |
| Test material | — | — | mdB-44 200 |
| (mg/kg BW) | | | |
| Glucose (mg/dL) | 149.0 ± 10.8 | 196.8 ± 7.8** | 185.2 ± 7.5 |
| Insulin (ng/mL) | 2.62 ± 0.21 | 9.12 ± 0.97*** | 5.67 ± 0.81^{#} |
| HOMA-IR | 22.9 ± 2.2 | 107.1 ± 12.4*** | 62.3 ± 9.3^{##} |
| QUICKI | 0.253 ± 0.002 | 0.218 ± 0.004*** | 0.230 ± 0.003^{#} |

Glucose was significantly increased in the high-fat diet control group (Group 2), compared to the control diet group (Group 1), and it was reduced in Group 3, compared to the high-fat diet control group (Group 2).

The blood insulin content was significantly increased as 9.12±0.97 ng/mL in the high-fat diet control group (Group 2), compared to 2.62±0.21 ng/mL in the control diet group (Group 1), while it was significantly reduced as 5.67±0.81 ng/mL in the mdB-44 administration group (Group 3), compared to the high-fat diet control group.

The insulin resistance was significantly increased as 107.1±12.4 in the high-fat diet control group (Group 2), compared to 22.9±2.2 in the control diet group (Group 1), while it was significantly reduced in the mdB-44 administration group (Group 3). The insulin sensitivity was significantly reduced as 0.218±0.004 in the high-fat diet control group (Group 2), compared to 0.253±0.002 of the control diet group (Group 1), while it was increased in the mdB-44 administration group (Group 3), compared to the high-fat diet control group. Through this, it was confirmed that the marmelo extract administration group inhibited insulin resistance caused by high-fat diet to improve impaired glucose tolerance.

6.4 Fat-Related Numerical Value Analysis

The contents of triglyceride (TG), total cholesterol (CHOL) and HDL-cholesterol (HDL-CHOL) in blood collected in Example 6.1 were measured using a blood biochemical analyzer (KoneLab 20 XT, Thermo Fisher Scientific), and using the blood cholesterol content and HDL-cholesterol content, an atherogenic index was calculated by the following Equation 4.

Atherogenic index=total cholesterol content/HDL-cholesterol content [Equation 4]

The obtained result was shown in Table 12.

TABLE 12

| | Group 1 | Group 2 | Group 3 |
|---|---|---|---|
| Experiment diet | CD | HFD | HFD |
| Test material | — | — | mdB-44 200 |
| (mg/kg BW) | | | |
| TG (mg/dL) | 77.1 ± 3.3 | 90.4 ± 3.4^{*} | 77.3 ± 3.2^{#} |
| CHOL (mg/dL) | 165.7 ± 8.4 | 188.4 ± 4.7^{*} | 171.8 ± 5.3^{#} |
| HDL-CHOL | 151.6 ± 6.2 | 126.9 ± 5.8^{**} | 150.9 ± 6.1^{#} |
| (mg/dL) | | | |
| Atherogenic | 1.09 ± 0.04 | 1.52 ± 0.11^{**} | 1.15 ± 0.04^{##} |
| index | | | |

In general, when a high-fat diet is ingested, the supple of fatty acids increases and the concentration of triglycerides and total cholesterol in blood increases, while the concentration of HDL-cholesterol in blood tends to decrease, which causes cardiovascular and coronary vascular disease.

The contents of blood triglycerides and blood total cholesterol were significantly increased in the high-fat diet control group (Group 2), compared to the control diet group (Group 1), while it was significantly reduced in the Group 3 to which mdB-44 was administered, compared to Group 2, and in particular, the blood triglyceride numerical value was measured very similarly to the control diet group (Group 1). The blood HDL-cholesterol content was significantly reduced in the high-fat diet control group (Group 2) compared to the control diet group (Group 1), while it was significantly increased in Group 3 to which mdB-44 was administered, compared to Group 2, and was measured very similarly to the control diet group (Group 1). As a result of calculating the atherogenic index (See Equation 4), which is the most effective predictive factor of the occurrence of coronary artery disease using the blood cholesterol content and HDL-cholesterol content, it was significantly increased as 1.52±0.11 in the high-fat diet control group (Group 2), compared to 1.09±0.04 of the control diet group (Group 1), while it was significantly reduced as 1.15±0.04 (Group 3) in the mdB-44 200 mg/kg BW administration group (Group 3), compared to Group 2.

6.5 Serum Leptin and Adiponectin Content Analysis

Adipose tissue is recognized as an endocrine organ that not only stores surplus energy, but also affects the metabolic process of the whole body by synthesizing and secreting various adipokines that play a very important physiological role. Leptin, one of the representative adipokines secreted from adipose tissue, is an important substance that regulates energy intake and storage, insulin sensitivity, and metabolic rate, and it is known that the secretion of leptin increases in case of obesity. Another major adipokine, adiponectin, is known as an important hormone that increases fatty acid oxidation in muscle and at the same time, prevents chronic disease such as diabetes and metabolic syndrome through the role of anti-inflammatory cytokines.

The leptin and adiponectin contents in blood of the blood collected in Example 6.1 were measured using a measurement kit purchased from R&D Systems according to the method suggested by the manufacturer, and the result was shown in Table 13 below.

TABLE 13

| | Group 1 | Group 2 | Group 3 |
|---|---|---|---|
| Experiment diet | CD | HFD | HFD |
| Test material | — | — | mdB-44 200 |
| (mg/kg BW) | | | |
| Adiponectin | 10.45 ± 0.26 | 9.45 ± 0.25* | 10.84 ± 0.37^{##} |
| (ng/mL) | | | |
| Leptin (ng/mL) | 21.14 ± 2.96 | 116.04 ± 9.49*** | 69.53 ± 5.13^{###} |

The blood leptin content was significantly increased in the high-fat diet control group (Group 2) compared to the control diet group (Group 1), and this was significantly reduced in the mdB-44 administration group (Group 3) compared to Group 2. The blood adiponectin content was significantly reduced in the high-fat diet control group (Group 2) compared to the control diet group (Group 1), and it was increased in the mdB-44 administration group (Group 3), compared to the high-fat diet control group (Group 2). It is known that the increase of the size, number and amount of fat cells in the visceral fat tissue has a positive correlation with the amount of leptin secretion, and a negative correlation with the amount of adiponectin secretion, and therefore, by the above result, the mdB-44 administration group significantly reduced the increased blood leptin and increased the amount of adiponectin secretion in obesity mice to show anti-obesity effect.

6.6 Histological Analysis of Epididymal Fat Tissue

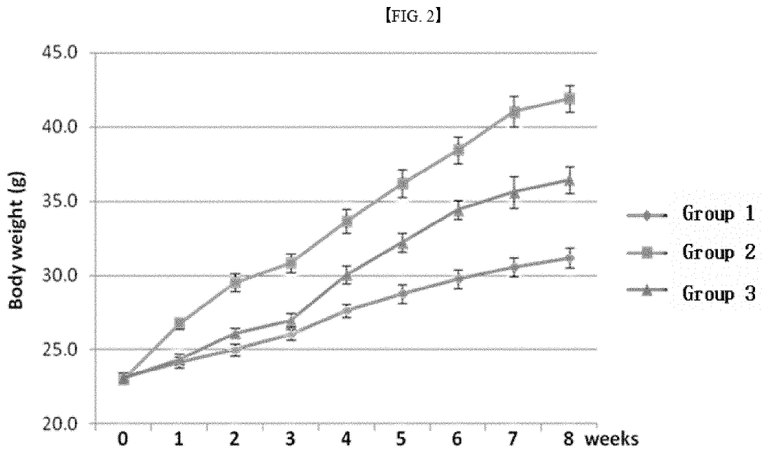

The fixed epididymal fat tissue fixed by removing from the experimental animal in Example 6.1 was embedded in paraffin and a tissue section of 5 μm was prepared from the embedded tissue. After paraffin removal, tissue was hydrated by decreasing % of alcohol sequentially from 100% alcohol to 0% alcohol (H₂O). Then, the tissue was stained (hematoxylin & eosin staining, H&E staining) using Accustain® Hematoxylin and Eosin Stains (Sigma-Aldrich Co.) according to the method suggested by the manufacturer, and the histological change of each tissue was observed using an optical microscope (Carl Zeiss). In addition, the fat cell size and cell number were measured using AxioVision Imaging analysis System for the photograph of epididymal fat tissue obtained by H&E staining. The histological change of the epididymal fat tissue was shown in FIG. 3, and the cell number of the measured fat tissue according to the fat cell size was shown in Table 14.

TABLE 14

| | Group 1 | Group 2 | Group 3 |
|---|---|---|---|
| Experiment diet | CD | HFD | HFD |
| Test material | — | — | mdB-44 200 |
| (mg/kg BW) | | | |
| <80 μm | 161.0 ± 7.1 | 1.0 ± 1.0*** | 41.7 ± 2.2^{###} |
| 80~100 μm | 16.0 ± 1.7 | 9.3 ± 1.5* | 45.0 ± 3.5^{###} |
| 100~120 μm | 0.0 ± 0.0 | 28.0 ± 4.0* | 12.7 ± 1.2^{#} |
| >120 μm | 0.0 ± 0.0 | 12.7 ± 1.2** | 0.0 ± 0.0^{##} |
| Fat cell number in same | 177.0 ± 8.7 | 51.0 ± 5.7*** | 99.3 ± 5.2^{##} |
| area | | | |
| Average fat globule size | 80.9 ± 0.1 | 108.5 ± 1.0*** | 88.3 ± 0.4^{###} |
| (μm) | | | |

(The numerical value is expressed as mean ± standard error, and means
*p < 0.05,
**p < 0.01,
***p < 0.001,
^{#}p< 0.05,
^{##}p< 0.01,
^{###}p < 0.001)

As shown in FIG. 3, the size of fat cells was significantly increased in the epididymal fat tissue of the high-fat diet control group (Group 2) compared to the control diet group (Group 1), and in the mdB-44 administration group (Group 3), the size of fat cells showed a tendency to decrease.

In addition, as shown in Table 14 showing the result of measuring the cell number according to the fat cell size of the epididymal fat tissue, it was confirmed that the size of fat cells was generally small, as in the control diet group (Group 1), the number of cells with a fat cell size of <80 μm was 161.0±7.1, while the number of fat cells with a size of >100 μm was not observed. It was confirmed that the size of fat cells in the high-fat diet control group (Group 2) was large as the number of cells with a fat cell size of 100˜120 μm was 28.0±4.0 and the cell number with a size of >120 μm was 12.0±2.1. As the result of comparing the cell number by fat cell size of the test material administration group (Group 3), compared to the high-fat diet control group (Group 2), the fat cell number with a size of <80 μm and a size of 80˜100 μm was significantly increased, and the cell number with a size of 100˜120 μm was significantly reduced. The fat cell number with a size of >120 μm was not observed in the mdB-44 200 mg/kg BW administration group (Group 3).

The total number of fat cells relative to the same area was significantly reduced as 51.0±5.7 in the high-fat diet control group (Group 2), compared to 177.0±8.7 of the control diet group (Group 1). For the average fat globule size, compared to 80.9±0.1 μm of the control diet group (Group 1), the fat globule size was significantly increased as 108.5±1.0 μm in the high-fat diet control group (Group 2), and the fat globule was significantly reduced as 88.3±0.4 μm (Group 3) in the mdB-44 200 mg/kg BW administration group (Group 3).

From the result, it could be confirmed that administration of the marmelo extract showed anti-obesity effect, and in particular, also had an effect of inhibiting hypertrophy of white fat tissue caused by high-fat diet.

6.6 Analysis of mRNA Expression of Energy Metabolism-Related Genes in Fat Tissue

After isolating total RNA using TRIzol® Reagent (Thermo Fisher Scientific) from the epididymal fat tissue obtained in Example 6.1, it was qualified using a micro-volume spectrophotometer (BioSpec-nano, SHIMADZU), and RNA with an OD260/280 value of 1.8 was used in the experiment. After obtaining cDNA using HyperScript™ RT master mix kit (GeneAll Biotechnology) from total RNA (2 μg), real-time PCR was performed using Rotor-Gene 300 PCR (Corbett Research) and Rotor-Gene™ SYBR Green kit (QIAGEN). The primer information used in the experiment was shown in Table 15. The quantitative analysis of expression of genes was performed using Rotor-Gene 6000 Series System Software program (Corbett Research).

TABLE 15

| | Sequence | | SEQ |
|---|---|---|---|
| mRNA | (5′ → 3′) | Genebank No. | ID NO |
| C/EBPα-FOR | TGGACAAGAACA | XM_021168520.2 | 1 |
| primer | GCAACGAGTAC | | |
| C/EBPα-REV | GCAGTTGCCCAT | XM_021168520.2 | 2 |
| primer | GGCCTTGAC | | |
| PPARγ-FOR | CAAAACACCAGT | XM_021164279.2 | 3 |
| primer | GTGAATTA | | |
| PPARγ-REV | ACCATGGTAATT | XM_021164279.2 | 4 |
| primer | TCTTGTGA | | |
| SREBP-1c-FOR | CACTTCTGGAGA | NM_011480.4 | 5 |
| primer | CATCGCAAAC | | |
| SREBP-1c-REV | ATGGTAGACAAC | NM_011480.4 | 6 |
| primer | AGCCGCATC | | |
| CPT1β-FOR | GTGCTGGAGGTG | NM_009948.2 | 7 |
| primer | GCTTTGGT | | |
| CPT1β-REV | TGCTTGACGGAT | NM_009948.2 | 8 |
| primer | GTGGTTCC | | |
| FAS-FOR | AGGGGTCGACCT | NM_007988.3 | 9 |
| primer | GGTCCTCA | | |
| FAS-REV | GCCATGCCCAGA | NM_007988.3 | 10 |
| primer | GGGTGGTT | | |
| aP2-FOR | GGATTTGGTCAC | NM_024406.3 | 11 |
| primer | CATCCGGT | | |
| aP2-REV | TTCACCTTCCTG | NM_024406.3 | 12 |
| primer | TCGTCTGC | | |
| HSL-FOR | CCGTTCCTGCAG | XM_030242181.1 | 13 |
| primer | ACTCTCTC | | |
| HSL-REV | CCACGCAACTCT | XM_030242181.1 | 14 |
| primer | GGGTCTAT | | |
| GAPDH-FOR | AGGTTGTCTCCT | XM_029478683.1 | 15 |
| primer | GCGACT | | |
| GAPDH-REV | TGCTGTAGCCGT | XM_029478683.1 | 16 |
| primer | ATTCATTGTCA | | |
| ACC1-FOR | GGAGATGTACGC | AY451393.1 | 17 |
| primer | TGACCGAGAA | | |
| ACC1-REV | ACCCGACGCATG | AY451393.1 | 18 |
| primer | GTTTTCA | | |
| ACL-FOR | TGGATGCCACAG | BC056378.1 | 19 |
| primer | CTGACTAC | | |
| ACL-REV | GGTTCAGCAAGG | BC056378.1 | 20 |
| primer | TCAGCTTC | | |

The effect of the test material on lipogenesis, degradation and energy metabolism-related mRNA expression in the epididymal fat tissue was analyzed, and the result was shown in Table 16.

TABLE 16

| | Group 1 | Group 2 | Group 3 |
|---|---|---|---|
| Experiment diet | CD | HFD | HFD |
| Test material | — | — | mdB-44 200 |
| (mg/kg BW) | | | |
| C/EBPα | 1.00 ± 0.36 | 4.10 ± 0.05*** | 2.20 ± 0.48^{##} |
| PPARγ | 1.00 ± 0.12 | 2.25 ± 0.20** | 1.11 ± 0.06^{##} |
| SREBP-1c | 1.00 ± 0.12 | 2.41 ± 0.23** | 1.51 ± 0.17^{#} |
| CPT1 | 1.00 ± 0.19 | 0.37 ± 0.07* | 2.28 ± 0.14^{###} |
| FAS | 1.00 ± 0.52 | 2.90 ± 0.39* | 0.25 ± 0.10^{###} |
| aP2 | 1.00 ± 0.11 | 1.98 ± 0.38* | 0.94 ± 0.13^{#} |
| HSL | 1.00 ± 0.07 | 0.45 ± 0.04*** | 0.78 ± 0.07^{##} |
| ACC1 | 1.01 ± 0.08 | 3.66 ± 0.42*** | 2.82 ± 0.35 |
| ACL | 0.96 ± 0.14 | 1.49 ± 0.15* | 0.75 ± 0.16^{#} |

(The result was calculated as the expression level of each material relative to the GAPDH expression level (expression level of each material/expression level of GAPDH), and is expressed as mean ± standard error, and means
*p < 0.05,
**p < 0.01,
***p < 0.001,
^{#}p < 0.05,
^{##}p < 0.01,
^{###}p < 0.001)

Transcriptional activation factors should be activated at the fat cell gene regulatory region for differentiation from preadipocytes to mature fat cells, and transcription factors which regulates differentiation of fat cells include SREBPs (sterol-regulatory element-binding protein), PPARs (Peroxisome Proliferator-Activated Receptor), C/EBPs (CCAAT/enhancer binding protein), and the like. SREBPs include SREBP-1 and SREBP-2, and among them, SREBP-1 is rapidly induced in the early stage of differentiation of preadipocytes to promote adipocyte differentiation and plays a role in promoting fat metabolism by increasing expression of genes related to fat metabolism.

As shown in Table 16, in the epididymal fat tissue, the SREBP-1c mRNA expression was significantly increased in the high-fat diet control group (Group 2), compared to the control diet group (Group 1), and it was significantly reduced in the mdB-44 200 mg/kg BW administration group (Group 3) compared to the high-fat diet control group (Group 2). C/EBPβ is expressed by induction of hormones (insulin, dexamethasone, etc.) in the early stage of adipocyte differentiation, and when C/EBPβ is activated, the expression of C/EBPα and PPARγ, the transcription factors of adipocytes, is induced in the late stage of differentiation. C/EBPα interacts with PPARγ to have an effect, and C/EBPα increases before expression of specific genes in adipose tissue to regulate energy homeostasis, and PPARγ is a major regulator of adipogenesis and is widely expressed in white adipose tissue. In the epididymal fat tissue, the C/EBPα and PPAR-γ mRNA expression was significantly increased in the high-fat diet control group (Group 2), compared to the control diet group (Group 1), and it was significantly reduced in the mdB-44 200 mg/kg BW administration group (Group 3) compared to the high-fat diet control group (Group 2).

FAS (fatty acid synthase) is an enzyme involved in fatty acid biosynthesis, and when fatty acids are synthesized in the liver, they are converted into triglycerides and play a role of circulating through blood vessels in the form of VLDL to deliver triglycerides to adipose tissue. The FAS mRNA expression in the epididymal fat tissue was significantly increased in the high-fat diet control group (Group 2), compared to the control diet group (Group 1), and it was significantly reduced in the mdB-44 200 mg/kg BW administration group (Group 3), compared to the high-fat diet control group (Group 2).

aP2, known as a target gene for lipogenesis, is expressed in fat cells and macrophages, and is known to be involved in inflammation and metabolic reactions. The aP2 mRNA expression in the epididymal fat tissue was significantly increased in the high-fat diet control group (Group 2), compared to the control diet group (Group 1), and the aP2 mRNA expression was significantly reduced in the mdB-44 200 mg/kg BW administration group (Group 3), compared to the high-fat diet control group (Group 2) (Table 16).

CPT-1 (Carnitine palmitoyl transferase-1) is a rate-limiting stage enzyme that inflows fatty acids into the mitochondria, and in order for fatty acids to flow into cells and be oxidized, a transferase inside the membrane is required, and CPT-1 is one of these transferases, and plays a major role in lipid use. The CPT-1 expression in the epididymal fat tissue was significantly reduced in the high-fat diet control group (Group 2), compared to the control diet group (Group 1), and the CPT-1 mRNA expression was significantly increased in the mdB-44 administration group (Group 3), compared to the high-fat diet control group (Group 2).

HSL (hormone-sensitive lipase) is distributed in various organs, but is mainly activated in adipose tissue, and becomes active HSL by secretion of hormones such as epinephrine and glucagon, and the active HSL plays a role of breaking down a triglyceride in lipoprotein into one glycerol and three fatty acids. The HSL mRNA expression in the epididymal fat tissue was significantly reduced in the high-fat diet control group (Group 2), compared to the control diet group (Group 1). The HSL mRNA expression was significantly increased in the mdB-44 200 mg/kg BW administration group (Group 3), compared to the high-fat diet control group (Group 2) (Table 16).

ACC1 is mainly present in adipose tissue, where fatty acid synthesis is important, and ACC2 is mainly present in oxidized tissue such as skeletal muscle and heart to regulate fatty acid synthesis and oxidation. The ACC1 mRNA expression in the epididymal fat tissue was significantly increased in the high-fat diet control group (Group 2), compared to the control diet group (Group 1), and the ACC1 mRNA expression was reduced in the mdB-44 200 mg/kg BW administration group (Group 3), compared to the high-fat diet control group (Group 2).

ACL is an enzyme involved in the synthesis of cytosolic acetyl-CoA by converting citrate to acetyl-CoA, and is used in important biosynthetic pathways such as fatty acids and cholesterol. The ACL mRNA expression in the epididymal fat tissue was significantly increased in the high-fat diet control group (Group 2), compared to the control diet group (Group 1), and the ACL mRNA expression was significantly reduced in the mdB-44 200 mg/kg BW administration group (Group 3), compared to the high-fat diet control group (Group 2).

6.7 Analysis of Protein Expression in Fat Tissue

In order to investigate the protein expression in the epididymal fat tissue, after adding lysis buffer (Pierce® IP Lysis buffer, Thermo Scientific) to the epididymal fat, it was homogenized with a homogenizer. The homogenized solution was centrifuged at 12,000 rpm for 10 minutes, and then the supernatant was collected to obtain epididymal fat tissue lysates. The protein amount of the tissue lysate was measured using BCA protein assay kit (Thermo Scientific).

After isolating protein (50 μg) by 10% sodium dodecyl sulfate polyacrylamide gel electrophoresis (SDS-PAGE), it was moved to polyvinylidene difluoride membrane (Milipore). After blocking the membrane in 5% skim milk-TBST (20 mmol/L Tris.HCl, pH 7.5, 150 mmol/L NaCl, 0.1% Tween 20) for 1 hour, an antibody to be measured was added respectively, and was stirred at 4° C. for 16 hours or at a room temperature for 1 hour. The information of the antibodies used in the experiment was shown in Table 17. Then, horseradish peroxidase (HRP)-linked anti-rabbit IgG or HRP-linked anti-mouse IgG was added and stirred for 1 hour, and each protein band was visualized by the enhanced chemiluminescence method using Luminata™ Forte Western HRP Substrate (Millipore). The protein expression level was quantified using ImageQuant™ LAS 500 imaging systems (GE Healthcare Bio-Sciences AB).

TABLE 17

| Antibody name | Detailed information | | Manufacturer |
|---|---|---|---|
| p-AMPK | Phospho-AMPKα (Thr172) | #2535 | Cell Signaling Technology |
| AMPK | AMPKa | #2532 | Cell Signaling Technology |
| β-actin | Beta-actin | #3700 | Cell Signaling Technology |

AMP-activated protein kinase (AMPK) is a factor that improves insulin resistance, and the activated AMPK is known to block ATP consumption pathways such as fat and cholesterol synthesis, while activating ATP production pathways such as glycolysis or fatty acid oxidation. In particular, in relation to lipid metabolism, it has been reported that it plays an important role in reducing body fat due to increased fatty acid oxidation, by inhibiting the activity of acetyl-CoA carboxylase (ACC) enzyme through protein phosphorylation.

In order to investigate the effect of the test material on AMPK activity in the epididymal adipose tissue, western blot was performed and quantified using the epididymal adipose tissue lysate, and the result was shown in Table 18.

TABLE 18

| | Group 1 | Group 2 | Group 3 |
|---|---|---|---|
| Experiment diet | CD | HFD | HFD |
| Test material | — | — | mdB-44 200 |
| (mg/kg BW) | | | |
| p-AMPK/AMPK | 1.00 ± 0.0 | 0.64 ± 0.04* | 1.50 ± 0.22^{#} |
| Ratio | | | |

(The numerical value is expressed as mean ± standard error, and means
*p < 0.05,
**p < 0.01,
***p < 0.001,
^{#}p < 0.05,
^{##}p < 0.01,
^{###}p < 0.001)

As shown in Table 18, as the result of evaluating the AMPK activity by the ratio of p-AMPK/AMPK, the AMPK activity reduced in the high-fat diet control group (Group 2) was significantly increased in the mdB-44 200 mg/kg BW administration group (Group 3).

As confirmed above, it was confirmed that the marmelo extract administered together with a high-fat diet effectively inhibited the increase in the body weight, liver and fat tissue weight, blood cholesterol, blood insulin and leptin contents, induced by the high-fat diet, and inhibited the size increase of fat cells. This suggests that the marmelo has anti-obesity effect and lipid improvement effect on dietary obesity, and furthermore, it has effects of prevention, treatment and/or improvement on metabolic disease, and suggests the potential to be developed as a health functional material with anti-obesity efficacy and/or effect of prevention, treatment and/or improvement on metabolic disease in the future.

### Example 7. Confirmation of Lipid Accumulation Inhibitory Effect According to Extraction Method

7.1 Cell Culture

Mouse-derived preadipocytes, 3T3-L1 cells, were purchased from the Korea Cell Line Bank, and used in the following examples to test the effect of inhibiting fat accumulation. The 3T3-L1 cells were cultured in a humidified CO₂ incubator (5% CO₂/95% air) at 37° C., using a cell culture medium (complete DMEM culture) prepared by adding 10% bovine calf serum (BCS), 100 units/mL penicillin and 100 μg/mL streptomycin to Dulbecco's Modified Eagle's Medium (DMEM, Welgene). When the cells reached about 80% full of the culture dish, the cell monolayer was washed with phosphate buffer saline (PBS, pH 7.4) and then the cells were subcultured by adding trypsin-2.65 mM EDTA, and the medium was changed every 2 days.

7.2 Cell Differentiation Induction and Test Material Treatment

The 3T3-L1 cells cultured in Example 7.1 were inoculated in a 24-well plate at a concentration of 1×10⁵ cells/well. After the cells reached confluence state, the cells were cultured by sequentially exchanging the cell culture medium with three types of differentiation-inducing medium (DM), to induce differentiation into adipocytes. Specifically, the differentiation was stimulated for 2 days by exchanging the cell culture medium with a differentiation-inducing medium obtained by adding DMI (1 μM dexamethasone, 0.5 mM 3-isobutyl-1-methylxanthine (IBMX), 5 μg/mL insulin) to 10% FBS-supplemented DMEM medium. After 2 days, the medium was exchanged with fresh differentiation-inducing medium obtained by adding 5 μg/mL insulin to 10% FBS-supplemented DMEM medium, to stimulate differentiation for another 2 days. After stimulation of differentiation for 4 days in total, cells were maintained in 10% FBS-supplemented DMEM medium for about 4 to 8 days to induce differentiation into adipocytes, during which the cell culture medium was exchanged every 2 days.

In order to test effects of marmelo extract on differentiation into adipocytes, the powder phase-marmelo extract (mdB-44) prepared in Example 1 was added to the differentiation-inducing medium at a concentration of 200 ug/ml. In addition, for comparison, an ethanol extract (EE) of marmelo fruit (LICORICE EXTRACT (Tashkent, Republic of Uzbekistan)) was prepared by the same manner of Example 1 except that the marmelo fruit was not subjected to compression and juice removal, and used for the aforementioned test of adipocyte-differentiation at a concentration of 200 ug/ml. In addition, a control group in which culture and differentiation was induced without adding an extract and a control group in which differentiation was not performed without adding an extract were prepared.

7.3 Measurement of Adipocyte Differentiation (Fat Accumulation)

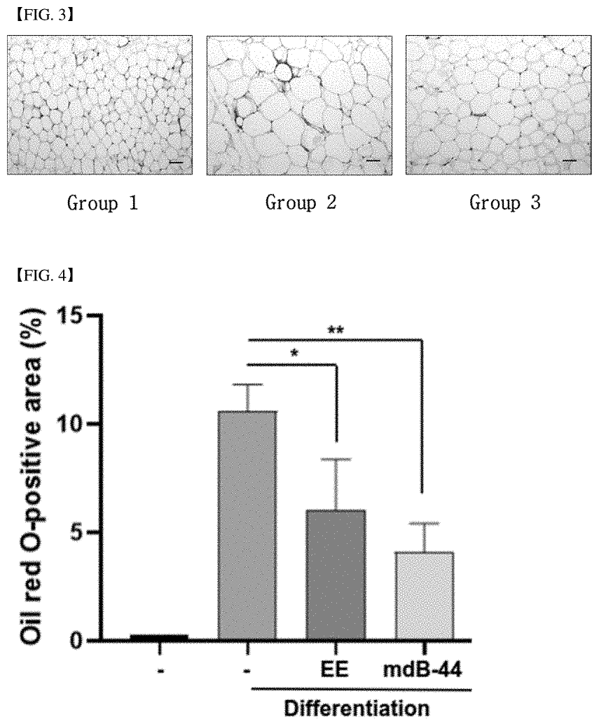

After performing induction of 3T3-L1 cell differentiation and treatment with the extract as in Example 7.2, the adipocyte-differentiated cells were rinsed with Dulbecco's phosphate buffered saline (Welgene), and fixed by adding 4% paraformaldehyde (Biosesang) at room temperature for 1 hour. After fixing, the cells were stained by treating with Oil Red 0 (Sigma-Aldrich) solution at room temperature for 1 to 2 hours. After observing the degree of staining of adipocytes, the cells were rinsed with distilled water and adipocytes were observed under a microscope. The degree of adipocyte differentiation was analyzed using the image J program, and the results are shown in FIG. 4.

7.4 Result

As shown in FIG. 4, it was confirmed that the extract (mdB-44) extracted from marmelo fruit subjected to compression and juice removal as described in Example 1 exhibits excellent lipid accumulation inhibitory effects compared to the ethanol extract (EE) extracted from marmelo fruit which was not subjected to compression and juice removal.

1. A method for preventing, improving or treating obesity, or reducing body weight or body fat, comprising administering a pharmaceutically effective amount of marmelo extract to a subject in need of prevention, improvement or treatment of obesity.

2. The method for preventing, improving or treating obesity, or reducing body weight or body fat according to claim 1, wherein the administering is administering the marmelo extract in an amount of 1 to 400 mg/kg, based on the body weight of the subject to be administered.

3. The method for preventing, improving or treating obesity, or reducing body weight or body fat according to claim 1, wherein the administering is administering the marmelo extract in an amount of 0.0048 to 1.92 g/day, based on the body weight 60 kg of the subject to be administered.

4. The method for preventing, improving or treating obesity, or reducing body weight or body fat according to claim 1, wherein the subject is a primate, a rodent, a mammal, a bird, a reptile, an amphibian or a vertebrate.

5. The method for preventing, improving or treating obesity, or reducing body weight or body fat according to claim 1, wherein the marmelo extract is administered by oral administration, intravenous administration, intramuscular administration, subcutaneous administration or intraperitoneal administration.

6. The method for preventing, improving or treating obesity, or reducing body weight or body fat according to claim 1, wherein the marmelo extract is, an extract obtained by extracting marmelo with 10 to 70 (v/v) % alcohol.

7. The method for preventing, improving or treating obesity, or reducing body weight or body fat according to claim 1, wherein the marmelo of the marmelo extract is, in a solid form remaining after removing marmelo juice after compressing marmelo.

8. The method for preventing, improving or treating obesity, or reducing body weight or body fat according to claim 6, wherein the alcohol extraction is performed by adding 1 to 10 volume times of alcohol at 50 to 100° C. and extracting for 1 to 24 hours.

9. A method for preventing, improving or treating metabolic disease, or lowering blood glucose or lowering cholesterol in blood, comprising administering a pharmaceutically effective amount of marmelo extract to a subject in need of prevention, improvement or treatment of metabolic disease.

10. The method for preventing, improving or treating metabolic disease, or lowering blood glucose or lowering cholesterol in blood according to claim 9, wherein the administering is administering the marmelo extract in an amount of 1 to 400 mg/kg, based on the body weight of the subject to be administered.

11. The method for preventing, improving or treating metabolic disease, or lowering blood glucose or lowering cholesterol in blood according to claim 9, wherein the administering is administering the marmelo extract in an amount of 0.0048 to 1.92 g/day, based on the body weight 60 kg of the subject to be administered.

12. The method for preventing, improving or treating metabolic disease, or lowering blood glucose or lowering cholesterol in blood according to claim 9, wherein the subject is a primate, a rodent, a mammal, a bird, a reptile, an amphibian or a vertebrate.

13. The method for preventing, improving or treating metabolic disease, or lowering blood glucose or lowering cholesterol in blood according to claim 9, wherein the marmelo extract is administered by oral administration, intravenous administration, intramuscular administration, subcutaneous administration or intraperitoneal administration.

14. The method for preventing, improving or treating metabolic disease, or lowering blood glucose or lowering cholesterol in blood according to claim 9, wherein the marmelo extract is,
an extract obtained by extracting marmelo with 10 to 70 (v/v) % alcohol.

15. The method for preventing, improving or treating metabolic disease, or lowering blood glucose or lowering cholesterol in blood according to claim 9, wherein the marmelo of the marmelo extract is,
in a solid form remaining after removing marmelo juice after compressing marmelo.

16. The method for preventing, improving or treating metabolic disease, or lowering blood glucose or lowering cholesterol in blood according to claim 14,
wherein the alcohol extraction is performed by adding 1 to 10 volume times of alcohol at 50 to 100° C. and extracting for 1 to 24 hours.

17. The method for preventing, improving or treating metabolic disease, or lowering blood glucose or lowering cholesterol in blood according to claim 9, wherein the metabolic disease is diabetes, hyperlipidemia, arteriosclerosis, hyperinsulinemia, or metabolic syndrome.
